(19) United States
(12) Patent Application Publication
(10) Pub. No.: US 2009/0305356 A1
(43) Pub. Date: Dec. 10, 2009

(54) METHODS AND APPARATUS FOR THE USE OF ULTRASONIC ENERGY TO IMPROVE ENZYMATIC ACTIVITY DURING CONTINUOUS PROCESSING

(75) Inventors: Susan E. Poniatowski, Westford, MA (US); John W. Walkinshaw, Westford, MA (US)

Correspondence Address: FOLEY & LARDNER LLP, 111 HUNTINGTON AVENUE, 26TH FLOOR, BOSTON, MA, 02199-7610, US

(73) Assignee: Paper Quality Management Associates

(21) Appl. No.: 12/476,010

(22) Filed: Jun. 1, 2009

Related U.S. Application Data
(60) Provisional application No. 61/059,501, filed on Jun. 6, 2008.

(51) Int. Cl. C12P 19/00; C12N 13/00

(52) U.S. Cl. 435/72; 435/173.2

(57) ABSTRACT

Described herein are methods and devices for increasing enzymatic activity during continuous processing by applying ultrasonic energy.

### RELATED APPLICATION

This application claims the benefit of prior provisional application Ser. No. 61/059,501, filed Jun. 6, 2009, the contents of which are hereby incorporated by reference in its entirety.

### BACKGROUND

Current methods to enhance enzyme action for converting paper into sugars involve the use of heat to increase batch temperatures and high speed mixing technology.

High-speed mixers and sheer mixing devices can be used to increase the physical action between the cellulosic materials and the enzymes. Increased physical contact opportunities and the ability of the sheer action of the mixer to break apart the fibrous cellulose materials can enhance enzyme activity. High-speed and sheer mixing devices act on the enzyme and the much larger quantities of cellulosic materials and water present. The majority of the energy is expended in moving cellulosic components and water rather than increasing the mobility of the enzyme within the suspended material. Mixing processes are frequently batch process operations that may not be easily integrated into continuous flow chemical processes such as those found in the paper, chemical or biofuels industry.

Using thermal energy to increase enzyme activity can denature enzymes, resulting productivity losses. Non-uniformity in process temperature can lead to a partial denaturing of the enzyme, resulting in inconsistent processing and productivity losses.

As there are practical and physical drawbacks to increasing enzyme activity in breaking down paper to basic sugars, new devices and methods for increasing enzyme efficiency are desirable.

### SUMMARY

The methods and devices utilize ultrasonic energies that impart a localized high energy mixing action to enzymes as they are moved about in a relatively stationary cellulose/water mixture. The application of ultrasound energy on the enzymes increases enzymatic activity on cellulose and produces faster, more efficient results than just mixing alone. An additional benefit from the use of ultrasonic action on the cellulosic material is that it increases the rate of hydration or swelling of the cellulosic materials. This results in increased accessibility of the enzymes to the inner core of wood and plant fibers, which leads to increased enzyme kinetics.

In one embodiment, a method for increasing enzymatic activity during a continuous processing reaction, includes applying ultrasonic energy to the reaction. In a particular embodiment, the enzymatic activity is derived from an enzyme selected from the group consisting of: cellulase, cellobiase and lignase. In a particular embodiment, the enzymatic activity is that of an enzyme expressed by a microorganism, e.g., a microorganism from a genus selected from the group consisting of: Trichoderma, Saccharomyces, Kluyveromyces, Dekkera, Candida, Aspergillus, Microbispora, Zymomonas, Chrysosporium, Escherichia, and Clostridium. In a particular embodiment, the microorganism is genetically modified, e.g., modified to expresses an exogenous enzyme. In one embodiment, the ultrasonic energy is applied continuously or in pulses. In one embodiment, the frequency of the applied ultrasonic energy is either fixed or cycled through a range of frequencies using a sweep oscillator.

In one embodiment, a method for improving the efficiency of the saccharification of cellulosic material during continuous processing, comprises administering ultrasonic energy to a saccharification reaction.

In one embodiment, a method for increasing enzyme mobility during a continuous process, comprises agitating the continuous process mixture using ultrasonic energy.

### BRIEF DESCRIPTION OF THE DRAWINGS

The patent or application file contains at least one drawing executed in color. Copies of this patent or patent application publication with color drawing will be provided by the Office upon request and payment of the necessary fee.

FIG. 1 is a graph of the raw data collected for the saccharification of toner printed waste paper using T. reesis enzymes with and without ultrasound. See Tables 1 and 3.

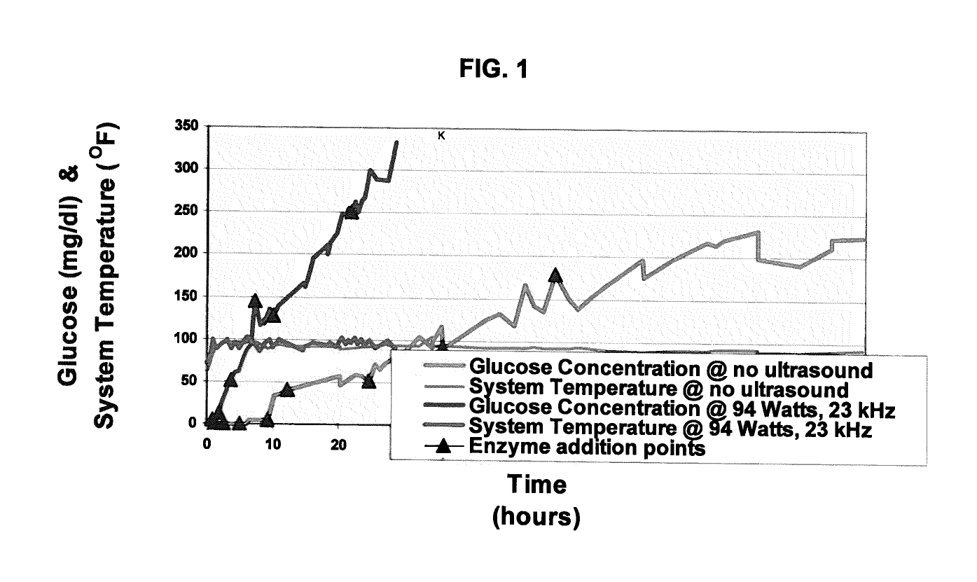

FIG. 2 is a comparison of the linear regression analysis of the data from FIG. 1 showing the increased rate of production of glucose with the addition of ultrasound energy. Reaction kinetics based on zero order reactions. See Table 2.

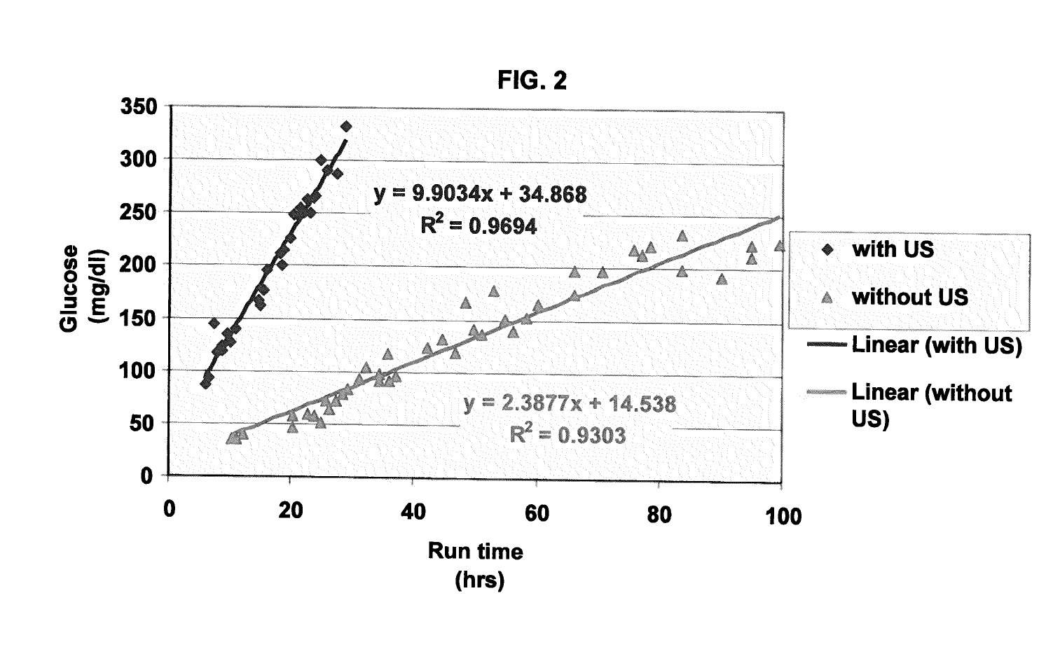

FIG. 3 is a graph showing the effects of ultrasound at different power levels on the saccharification of toner printed waste paper using T. reesis enzymes using a polynomial regression analysis through the origin of the no ultrasound rate. Applied power=total power−no load power.

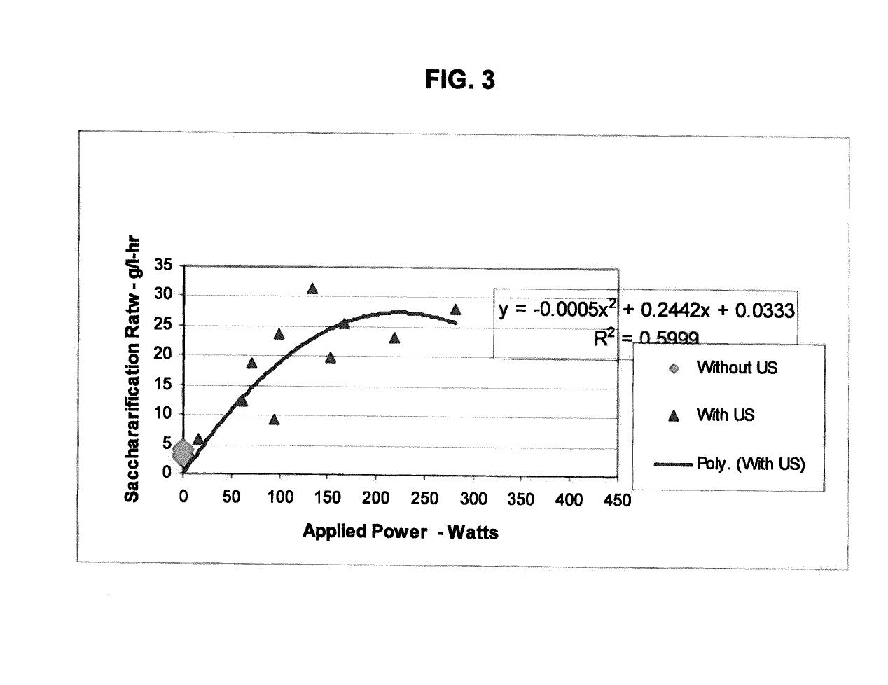

FIG. 4 is a graph showing the collected data for a single saccharification trial of toner printed waste paper using T. reesis enzymes using the continuous application of ultrasound at an applied power level of 94 watts. Enzyme addition total=14 mL. See Table 3.

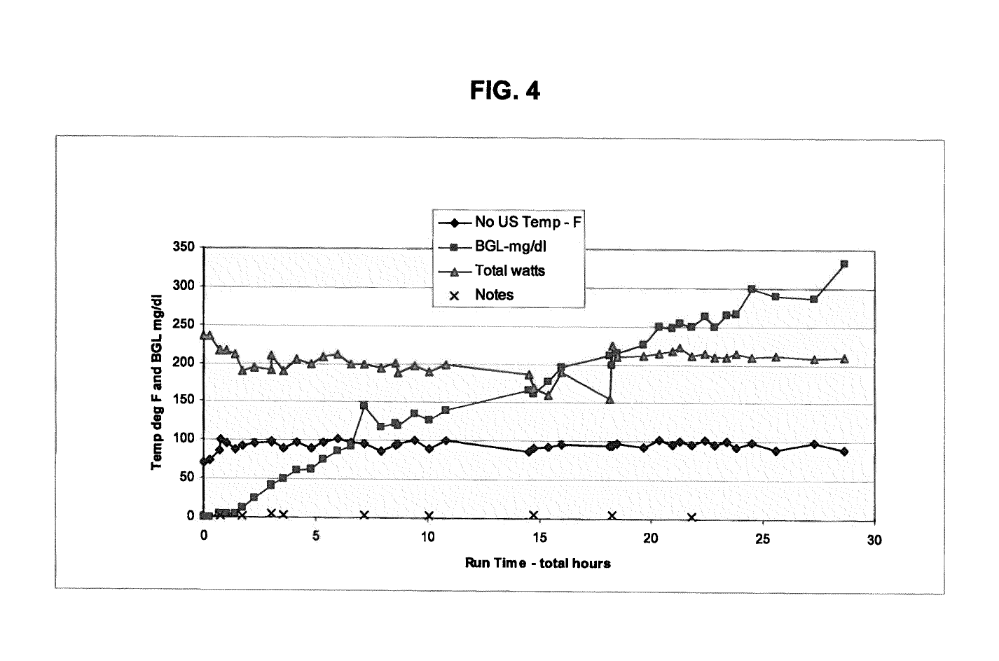

FIG. 5 is a graph of linear regression analysis of glucose production data from FIG. 4.

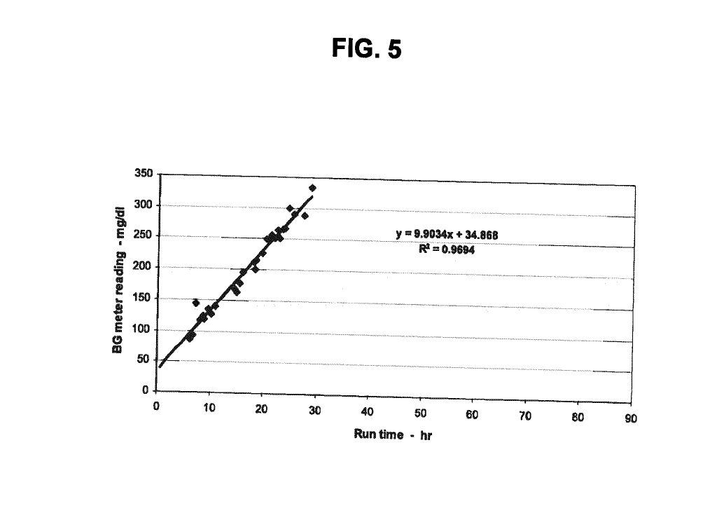

FIG. 6 is a graph showing the collected data for a single saccharification trial of toner printed waste paper using 7 reesis enzymes using a pulsed application of ultrasound at a peak applied power of 281 watts with an averaged applied power level of 165 watts. Approximate duty cycle of 75%. Enzyme addition total=10 mL. See Table 4.

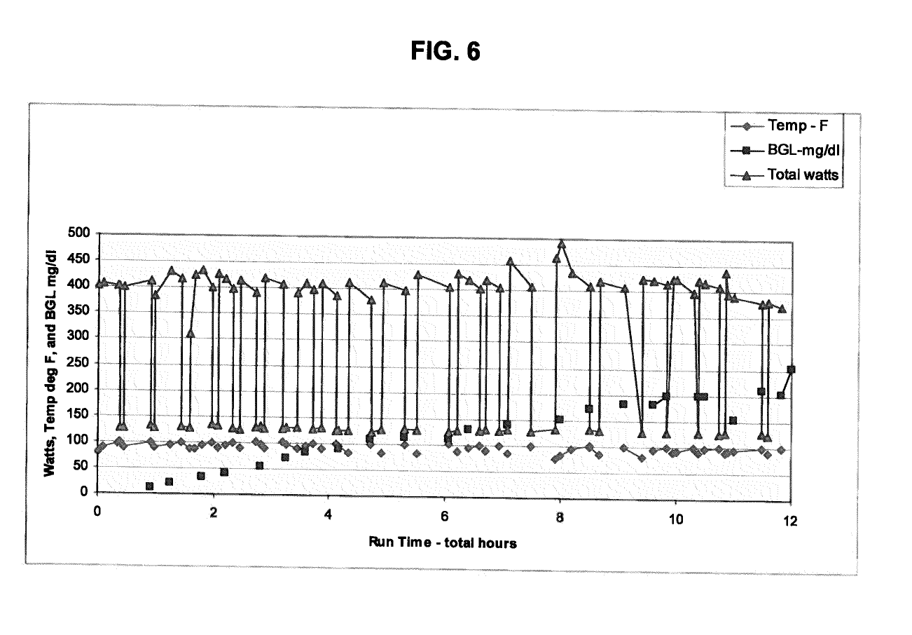

FIG. 7 is a graph of linear regression analysis of glucose production data from FIG. 6.

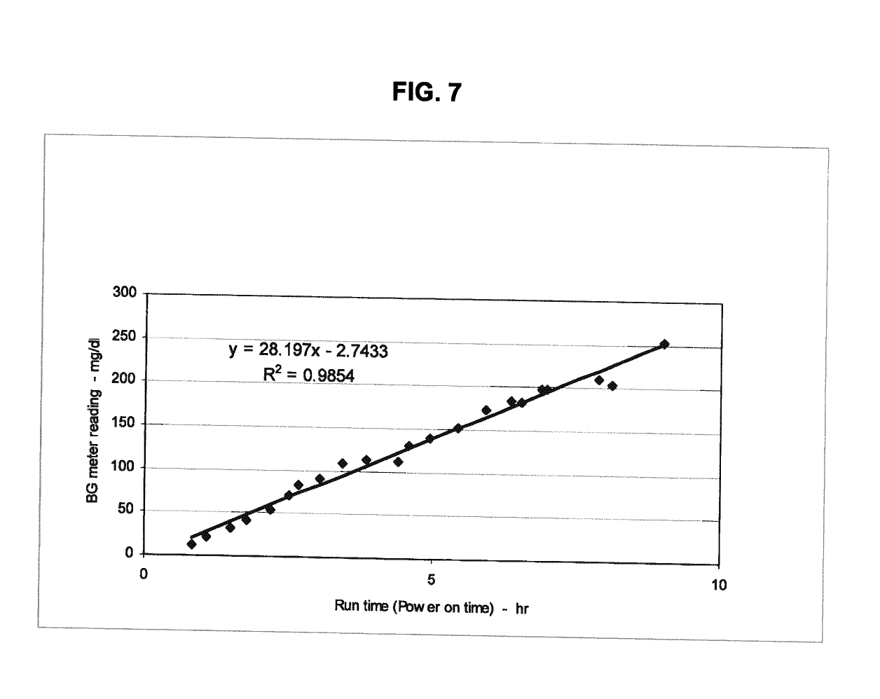

FIG. 8 is a graph showing the collected data for a single saccharification trial of toner printed waste paper using T. reesis enzymes using a higher power level at the start to open cellulose fiber structure to enzyme attack with a lowering of the power level near the end of the run. Total enzyme addition=10 mL. See Table 5.

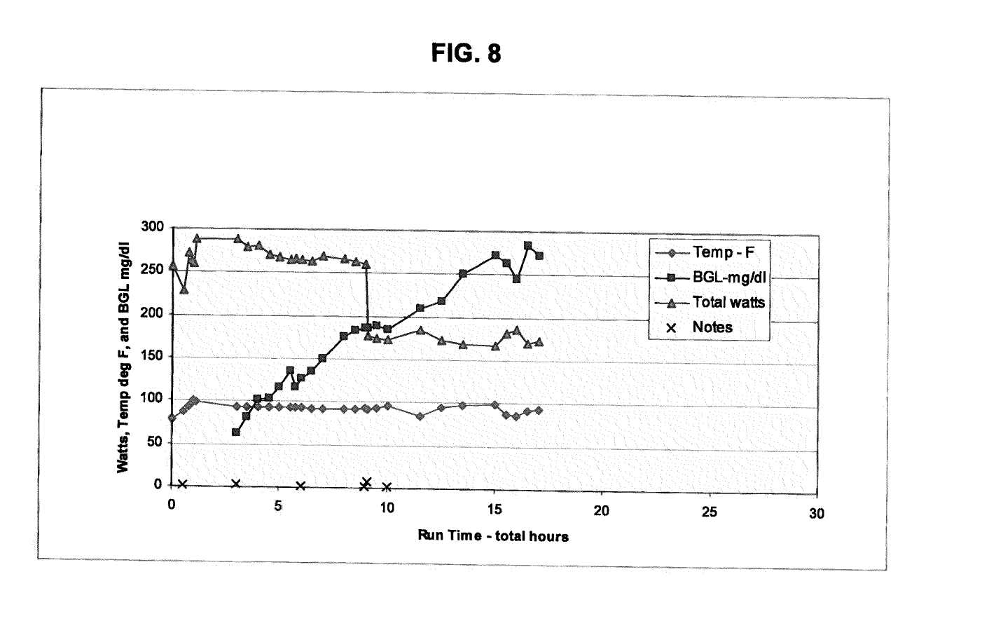

FIG. 9 is a graph of linear regression analysis of glucose production data from FIG. 6. Initial applied power level=153 watts yielding a glucose production rate of 19.7 mg/dL-hr and a final applied power level=61 watts for yielding a glucose production rate of 12.3 mg/dL-hr.

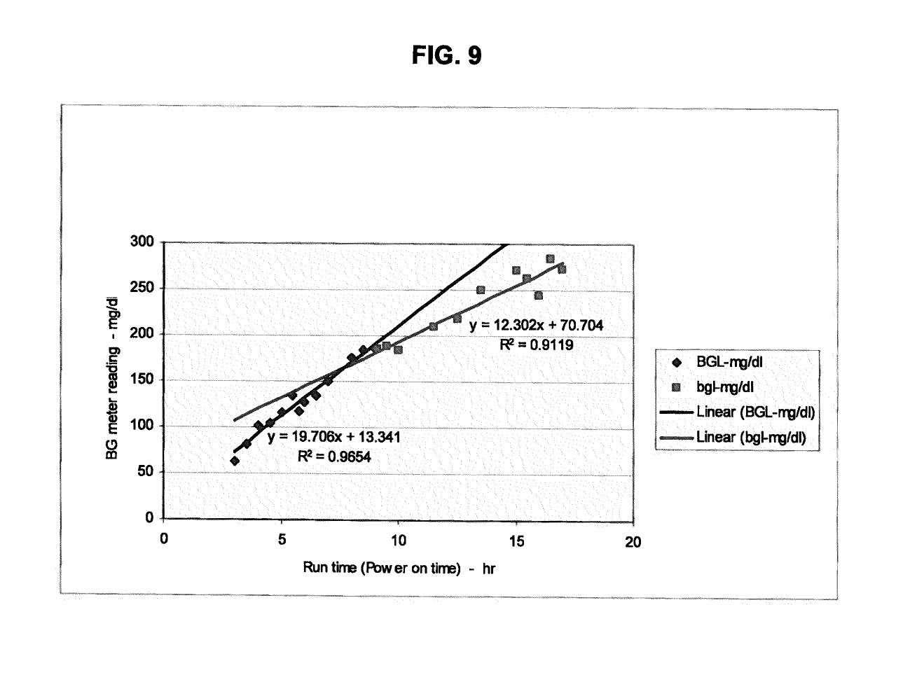

### DETAILED DESCRIPTION

The methods and devices are based on the unexpected finding that ultrasonic energy, can be applied to an enzymatic reaction mixture such that the applied ultrasonic energy improves enzyme activity. The applied ultrasonic energy, which can be applied either continuously or in pulses, is useful for improving enzyme activity for continuous process reactions. As used herein, “continuous processing” refers to processing in which new materials are added and products removed continuously at a rate that maintains a fixed volume.

The methods and devices described herein are suitable for all enzymatic reactions that can be kinetically enhanced by exposure to an ultrasonic field as a result of the increase in chemical activity resulting from ultrasonically produced pressure waves. These reactions would specifically included all reactions incorporating one or more enzymes, yeasts, microorganisms, viruses, catalysts, biocatalysts or chemicals resulting in products comprised of chemical structures present or not present in the entering feedstocks. Examples include, enzymes suitable for converting cellulosic material, e.g., municipal solid waste, paper and paper-related products, or any other source of cellulosic material, to basic sugars or other products (e.g., alcohols).

Ultrasonic energy can be applied to a reaction mixture during continuous processing at various frequencies suitable to increase enzyme activity. Frequencies of between about 23 kHz and about 24 kHz, between about 22 kHz and about 25 kHz, or between about 20 kHz and about 30 kHz can be used. Ultrasonic energy can be applied at a fixed (constant) frequency, or ultrasonic energy can be applied by cycling through a range of suitable frequencies using, for example, a sweep oscillator.

Enzymes suitable for the methods and devices described herein include, for example, enzymes used in the conversion of cellulose to glucose. Microorganisms often express enzymes suitable for the devices and methods. Such microorganisms can be used directly in a continuous processing reaction, or the enzymes can be isolated from the microorganisms. Suitable microorganisms can be grown in the presence of a suitable substrate, for example, during a continuous processing reaction, whereby the microorganismal enzymes act on the substrate during processing. The reaction mixture can comprise more than one microorganism. The reaction mixture can also be supplemented with isolated enzymes or enzymes contained in cellular extracts either alone or in combination with microorganisms. Some examples of suitable microorganisms and their expressed enzymes are described herein.

### Enzymes

Cellulases are a family of enzymes that can hydrolyze cellulose to glucose. Trichoderma viride, for example, contain T. viride cellulase capable of reducing cellulose to glucose. In addition to the cellulase content, T. viride are rich in cellobiase, an enzyme that reduces the cellobiose (a two chained cellulose unit) to glucose. When used alone, enzymes from T. viride operate more efficiently than when used in combination. To prevent enzyme denaturation, however, enzymes can be supplemented by a cellobiase enzyme source. Conversion of waste paper can be accomplished, for example, using enzymes from T. viride (NOVO-Celluclast 100L; Novozyme, Bagsvaerd, Denmark) with a supplemental enzyme (Novozyme 188; Novozyme, Bagsvaerd, Denmark). Enzymes from T. reesei are capable of reducing cellulose to glucose, however, this microorganism is lower in cellobiase content then T. viride. The T. reesei cellulose will stop functioning if attached to a cellubiose unit. Typically this enzyme is used with an enzyme of the cellobiase family to allow for continuous processing. This enzyme has been used as a single enzyme in the latest trials dealing with the effect of ultrasound energy.

In addition to naturally-occurring microorganisms and enzymes, microorganisms can be genetically modified and used in the methods and devices. For example, all naturally-occurring and genetically modified yeast strains of Saccharomyces cerevisiae are suitable for use in the methods and devices.

Cellobiases are a family of enzymes that can hydrolyze cellobiose (a molecule consisting of two glucose units) to glucose. Members of this family of enzyme are expressed by, for example, Aspergillus niger (Novozyme 188), thermophilic microorganism Microbispora bispora (Rutgers P&W cellulase and its mutants), Zymomonas mobilis (NRRL B-806 and its mutants), and Candida shehatae (NRRL Y-12858 and its mutants). Other examples of enzymes and the microorganisms that express them are known. Several commercially available enzymes can be found, for example, at the web site for Sigma Aldrich (sigmaaldrich.com/catalog/search/TablePage/15547879).

Glucosidase activity can also be enhanced by the methods and devices. For example, BGL5 polypeptides, recombinant or naturally-occurring, having a β-glucosidase activity can be used. BGL5 glucosidase can be isolated or expressed in an organism comprising bg15 nucleic acid sequences, which encode the polypeptides having beta-glucosidase activity. Nucleic acid constructs, vectors, and host cells comprising the nucleic acid sequences can be used to provide the β-glucosidase activity of BGL5.

Other industrial enzymes and microorganisms suitable for use in the methods and devices include, for example, endoglucanases and betaglucanases, e.g., expressed by Chrysosporium lucknowense, used in, for example, the nutrition, pulp and paper and pharmaceutical industries; Candida rugosa for the production of wax esters; and Candida bombicola for the production of sophorose lipids. Extensive lists of enzymes and sources can be found at the Sigma Aldrich web site (sigrnaaldrich.com/Area_of_Interest/Biochemicals/Enzyme_ExploreriAnalytical_Enzymes/Novozymes.html). Other such enzymes include, but are not limited to, for example, xylosidases, arabinofuranosidases, acetylxylanesterases, glucanases, mannases, endoglucanases, pectate lyases, cellobiohydrolase, pectin methyl esterases, arabinases, lignases, galactosidases, glucuronidase, xylanolytic-cellulolytic enzymes, A-L-arabinofuranosidase, xylanase, amylase and endoglucanase.

### Microbes

In addition to microorganisms that express enzymes suitable for use in the methods and devices, such microorganisms (or others) can be genetically engineered to express enzymes of interest. Escherichia coli, for example, expresses enzymes suitable for use in the present methods and devices, and can be further modified to express one or more exogenous enzymes (e.g., for the production of Glucosamine and N-acetylglucosamine via fermentation). Yeasts (e.g., Kluyveromyces, Candida molinschiana, Dekkera bruxellensis), both naturally-occurring and genetically modified strains, can be used, for example, for the production of alcohols and fine chemicals from cellulose-lignin feedstocks. Other useful microorganisms include extremophiles, which grow in otherwise harsh conditions (e.g., temperature extremes, pH extremes, etc.). One example is Candida antartica for the production of biodiesel fuel and biosurfactants, and the degradation of n-alkanes. It is also used as a biocatalyst for the asymmetric synthesis of amino acids/amino esters, due to its chemoselectivity towards amine groups.

Other microbes include, but are not limited to, for example, Clostridium (beijerinckii, acetobutylicum, thermocellum), Pichia stipitis, Aureobasidium pullulans, Mucor circinelloides, Fusarium (verticillioides, proliferatum), Saccharophagus degradans and Paenibacillus woosongensis.

### Biocatalysts

Biocatalysts, e.g., for production of the pharmaceutical intermediate (R,S)-1, cis-4-hydroxy-Dproline, 5-cyanovaleramide, the industrial solvent dimethyl-2-piperidone, and 3-hydroxyalkanoic acid for the synthesis of co-polyesterpolyols, are also suitable for the methods and devices.

### Butanol

Recombinant organisms including bacteria, cyanobacteria, filamentous fungi and yeasts can be engineered to have a 2-butanol biosynthetic pathway. The process involves providing a recombinant microbial host cell encoding a polypeptide that catalyzes conversion of pyruvate to 2-butanone by the 2-butanol biosynthetic pathway.

Other enzymes and microorganisms include, for example, N-methyl-N-nitro-N-nitrosoguanidine (NTG), Co-enzyme A transferase, Clostridium acetobutylicum and other Clostridium strains (e.g., C. tyrobutyricum, C. thermobutyricum, C. butyricum, C. cadaveros, C. cellobioparum, C. cochlearium, C. pasteurianum, C. roseum, C. rubrum, C. sporogenes, C. beijerinkii, C. aurantibutyricum and C. tetanomorphum).

### Cellulosic Materials

Cellulosic materials are used in many processes as a feed chemical. Cellulosic materials can be derived from any source of cellulose, e.g., plant or modified organisms (e.g., bacteria and yeast). Examples can be found in the paper industry where, for example, wood materials are used to make paper, cellulose materials are used as a feed stream to produce ethanol, and cotton is a material critical to the textile industry. Municipal solid waste is another source of available cellulosic material. The process feed typically consists of cellulosic material suspended in water. Enzymes or other chemicals are often added to the suspended cellulosic material to enhance, modify or convert the cellulose, either physically or chemically, to improve the quantity or quality of the resultant product.

General areas of application for improving the processing capacity of such reactions in the pulp and paper industries include the enzyme treatment of water suspensions of wood or plant fibers to enhance lignin and pitch removal, thereby reducing refiner energy demand, or in the treatment of recycled fiber, to improve the finished quality of paper. As described herein, the methods and devices are directed to the use of ultrasonic energy to improve enzyme activity in cellulosic processing reactions.

Ultrasonic energies on fiber suspensions have the advantage that the energy applied does not detract from fiber quality and can enhance paper strength. Additionally, in the biofuels industry, ultrasonic energy can be used to enhance the enzyme treatment of plant materials prior to fermentation and the production of ethanol.

Enzyme treatment of cellulosic material can be an expensive and relatively slow process. Typically heat is used to accelerate chemical reaction kinetics, but in the case of enzyme reactions, heat can quickly denature the enzymes causing them to loose functionality. To obtain faster treatment times process operators sometimes resort to increasing enzyme addition rates, thereby increasing enzyme costs. The methods and devices described herein can be used as an alternative method by which the enzyme reaction kinetics can be increased without denaturing the enzyme, reducing enzyme productivity or increasing enzyme addition levels.

The methods relate to the use of a device to accelerate the reduction of mixed office waste (MOW) paper into sugar using one or more enzymes, enhancing enzyme activity through the application of ultrasonic energies. The purpose of the methods and devices is to enhance the activity of enzymes on the treatment of the cellulosic materials passing through the unimpeded flow channel of the processing chamber wherein the material flowing through the chamber is subject to ultrasonic energies. Likewise, the methods and devices are useful for enhancing other chemical reactions such that the reactive components are contained within the chamber.

A useful device utilizes one or more ultrasonic transducers mounted on a hollow flow through chamber. The ultrasonic transducer can be made from, for example, polyvinylidene fluoride (PVDF) ultrasonic grade films, piezoelectric crystals or ceramics or other materials that produce vibration from the application of energy. With the correct length dimension of the piezoelectric transducer(s) and the centerline spacing between two or more transducers, the traducers act as a phased array, thus having the net effect of increased vibrational energy into the processing chamber (U.S. Pat. No. 6,736,904 Poniatowski et al. ('904), the contents of which are herein incorporated by reference in their entirety).

The processing chamber, designed in '904 as a resonant chamber, can be designed to process new and recycled paper pulp slurries. The ultrasonic vibrations are to “shake” toner image printing from the surface of recycled paper fibers and reduce in size toner print and other contraries found in paper pulp slurries—the smaller and or free contrary materials being easier to remove and hence produce a cleaner finished paper product. The ultrasonic transducers are mounted on the exterior surface of the flow-through chamber thus permitting unimpeded flow of the cellulosic-water suspension through the processing device.

Methods and devices suitable for achieving enhanced enzyme activity in cellulosic processing reactions and other chemical reactions on cellulosic materials can include, for example a device that can be used in a stand-alone configuration or applied to existing piping used to connect chemical processes equipment, the latter application being a benefit to existing process plants where space may be limited.

The methods and devices incorporate the use of predetermined length of transducer elements and spacing between the elements in a manner that permits the elements to be operated in a phased array, thereby increasing the effective ultrasonic energy entering the processing chamber. The use of multiple phased array transducers enhances the mixing rate. Existing devices with single or multiple transducers without consideration being given to the phasing of applied ultrasonic waves may operate with energy losses. Such an example of non-phased, multiple transducers is described in Mori et al., U.S. Pat. No. 3,946,829; and Kinley et al., U.S. Pat. No. 7,101,691; the contents of each of which are herein incorporated by reference in their entireties.

With proper design of transducer length and the centerline spacing between two or more transducers the flow chamber can be either a specially designed volume or can be a length of commercial tube manufactured of hard material such as, for example, metal, glass, ceramic, etc. This provides a means wherein the device can be used within a chemical plant either as a piece of “process equipment” or as a mounting on a section of normal process piping within the plant. Since the ultrasonic transducers are outside of the flow chamber they are not subjected to flow pressures, or chemical attack from corrosive or abrasive fluids within the process piping. This could be a major advantage in processes where special anti-corrosion materials must be used on wetted surfaces. Mounting the transducers on process piping provides a mechanism wherein this technology can be utilized in an existing plant where space between equipment is sometimes limited and additional process equipment will not fit in the space available.

An ultrasonic transducer can be mounted on the outside surface of the flow channel so as not to interfere with the flow of material through the processing chamber (U.S. Pat. No. 7,101,691 Kinley et al., outlines the use of internally mounted ultrasonic transducers.). This design feature becomes important as, when the concentration of cellulosic material increases over 0.5% (wt/wt) in solution, the suspended particles can “hang-up” or bridge across object(s) that protrude into the flow path. If this build-up or bridging occurs, then the flow channel can become completely blocked.

Currently under investigation in the biofuels industry is the use of simultaneous saccharification/fermentation reactions. For these applications, the use of the device would be particularly advantageous enhance the saccharification reaction (conversion of cellulose to glucose) while not compromising the longevity or activity of the yeast converting the glucose to ethanol. The device would not interfere with the flow of high solids biomass streams nor expend large amount of energy in bulk material mixing.

Bioprocessing from various industries are envisioned for the methods and devices. For the pulp and paper industry, for example, continuous processing by the methods described herein can be used for, inter alia, pitch and stickies removal, delignificaction in pulping processes, bleaching operations, reduced refining energy demand and cleaning and contrary removal in recycled pulps. For the biofuels industry, for example, continuous processing by the methods described herein can be used for, inter alia, saccharification of five and six numbered carbon carbohydrates to xylose and glucose. For chemical production, for example, continuous processing by the methods described herein can be used for, inter alia, nano-mixing of highly viscous materials.

### Exemplification

Example. Experimental Protocol for Enzyme Runs Using Ultrasonic Energy
Equipment: Processing tube 5GS-1.6×
Thick stock: Laser printed waste office papers pulped in a one gallon Waring Blender for ten minutes at a consistency of 100 grams a.d. paper per 3 L water.
Batch additives: Antibiotic (to retard bacterial growth)—solution of 25 mg of chloramphenicol dissolved in 1 mL 95% ethanol.
Buffer solution: 0.1 M citric acid solution in water.
Enzymes: Cellulase from Trichoderma reesei (ATCC 26921)
Batch preparation: 500 mL thick stock
   2 mL antibiotic
   15 mL buffer solution
   Water to dilute to 1500 mL

### Procedure

Add diluted stock to tube (tube will hold about 1300 mL)

Start circulating pump

Start ultrasonic generator:
Adjust frequency of operation 23.6±0.1 kHz
Adjust power level
Allow temperature to rise, or provide heat to circulation loop, until a temperature of 90+° F. is reached. Maintain temperature at 95±4° F.

Add 5 mL enzyme solution start run clock.

Additional enzymes to be added at the time intervals noted below;

| Run time (hr) | Addition (mL) |
|---|---|
| 2.5-3 | 2 |
| 5.5-6 | 1 |
| 8.5-9 | 1 |
| 11.5-12 | 1 |
| 14-15 | 1 |

Record operating data and glucose concentration as a function of time.

### Tables

TABLE 1

| | | |
|---|---|---|
| mixed | Glucose | graph |
| System | Concentration | enzyme |

| run time | Temperature | @ no | | addition |
|---|---|---|---|---|
| hr | @ no ultrasound | ultrasound | Notes | points |
| 0 | 62 | 0 | | |
| 0.17 | 67 | 0 | | |
| 0.75 | 82 | 0 | | |
| 0.92 | 85 | 0 | | |
| 1.17 | 88 | 0 | 1 | 0 |
| 1.34 | 90 | 0 | | |
| 1.42 | 92 | 0 | | |
| 1.67 | 92 | 0 | | |
| 2 | 95 | 0 | | |
| 2.59 | 96 | 0 | | |
| 2.67 | 96 | 0 | 2 | 0 |
| 2.92 | 97 | 0 | | |
| 3.26 | 97 | 0 | | |
| 3.42 | 97 | 0 | | |
| 3.92 | 97 | 0 | | |
| 4.42 | 96 | 0 | | |
| 4.84 | 96 | 0 | | |
| 4.92 | 96 | 0 | 2 | 0 |
| 5.5 | 95 | 0 | | |
| 6 | 96 | 0 | | |
| 6.5 | 96 | 5 | | |
| 6.59 | 99 | 5 | | |
| 6.67 | 93 | 5 | | |
| 6.84 | 94 | 5 | | |
| 6.89 | 93 | 5 | | |
| 7 | 93 | 5 | | |
| 7.5 | 96 | 5 | | |
| 9.17 | 89 | 5 | 2 | 5 |
| 10.17 | 94 | 35 | | |
| 11.17 | 94 | 36 | | |
| 12.17 | 92 | 40 | 3 | 40 |
| 20.17 | 91 | 58 | | |
| 20.25 | 88 | 46 | 4 | |
| 22.67 | 90 | 59 | | |
| 23.67 | 92 | 58 | | |
| 24.67 | 92 | 51 | 2 | 51 |
| 25.67 | 93 | 72 | | |
| 26.17 | 94 | 65 | | |
| 27.17 | 95 | 73 | | |
| 28.17 | 93 | 78 | | |
| 29.17 | 93 | 83 | | |
| 30.92 | 94 | 93 | | |
| 32.17 | 93 | 105 | | |
| 34.17 | 105 | 91 | | |
| 34.25 | 93 | 98 | | |
| 35.67 | 93 | 117 | 4 | |
| 35.84 | 93 | 91 | 2 | 91 |
| 36.92 | 93 | 96 | | |
| 42.17 | 91 | 124 | 4 | |
| 44.42 | 91 | 132 | | |
| 46.59 | 91 | 119 | | |
| 48.25 | 91 | 167 | | |
| 49.67 | 93 | 142 | | |
| 50.92 | 91 | 136 | | |
| 52.79 | 92 | 179 | 2 | 179 |
| 54.84 | 92 | 151 | | |
| 56.17 | 93 | 139 | | |
| 58.17 | 91 | 153 | | |
| 60.24 | 90 | 165 | 4 | |
| 66.04 | 88 | 198 | | |
| 66.17 | 88 | 175 | | |
| 70.67 | 90 | 198 | | |
| 75.75 | 90 | 218 | | |
| 77.17 | 91 | 214 | | |
| 78.37 | 91 | 221 | | |
| 83.59 | 91 | 232 | 4 | |
| 83.67 | 88 | 199 | | |
| 89.92 | 89 | 192 | | |
| 94.75 | 90 | 212 | | |
| 94.84 | 90 | 223 | | |
| 99.57 | 92 | 224 | | |
| average = | 92.80597015 | | | |

TABLE 2

| | with US | | without US |
|---|---|---|---|
| run time hr | BG mg/dl | run time hr | BG mg/dl |
| 6 | 87 | | |
| 6.58 | 93 | | |
| 7.17 | 144 | | |
| 7.92 | 117 | | |
| 8.58 | 123 | | |
| 8.67 | 119 | | |
| 9.42 | 135 | | |
| 10.08 | 127 | | |
| 10.83 | 140 | 10.17 | 35 |
| 14.53 | 167 | 11.17 | 36 |
| 14.75 | 162 | 12.17 | 40 |
| 15.42 | 177 | 20.17 | 58 |
| 16 | 196 | 20.25 | 46 |
| 18.17 | 212 | 22.67 | 59 |
| 18.25 | 200 | 23.67 | 58 |
| 18.5 | 215 | 24.67 | 51 |
| 19.67 | 226 | 25.67 | 72 |
| 20.35 | 249 | 26.17 | 65 |
| 20.93 | 248 | 27.17 | 73 |
| 21.3 | 255 | 28.17 | 78 |
| 21.83 | 250 | 29.17 | 83 |
| 22.38 | 263 | 30.92 | 93 |
| 22.83 | 250 | 32.17 | 105 |
| 23.38 | 265 | 34.17 | 91 |
| 23.83 | 267 | 34.25 | 98 |
| 24.5 | 300 | 35.67 | 117 |
| 25.58 | 290 | 35.84 | 91 |
| 27.33 | 288 | 36.92 | 96 |
| 28.67 | 333 | 42.17 | 124 |
| | | 44.42 | 132 |
| | | 46.59 | 119 |
| | | 48.25 | 167 |
| | | 49.67 | 142 |
| | | 50.92 | 136 |
| | | 52.79 | 179 |
| | | 54.84 | 151 |
| | | 56.17 | 139 |
| | | 58.17 | 153 |
| | | 60.24 | 165 |
| | | 66.04 | 198 |
| | | 66.17 | 175 |
| | | 70.67 | 198 |
| | | 75.75 | 218 |
| | | 77.17 | 214 |
| | | 78.37 | 221 |
| | | 83.59 | 232 |
| | | 83.67 | 199 |
| | | 89.92 | 192 |
| | | 94.75 | 212 |
| | | 94.84 | 223 |
| | | 99.57 | 224 |

TABLE 3

| Fiber | laser printed papers run in 1 gal Waring blender for several | |
|---|---|---|
| | minutes until fully dispersed | |
| Batch | 1400 ml total volume | |
| | 500 ml thick stock (100 g ad laser jet printed | |
| | paper/3000 ml water) | |
| | 5 ml enzyme to start | Enzyne added in increments - |
| | | see notes |
| | 15 ml 0.1 M citric acid buffer | |
| | water to dilute to 1500 ml | Water volume maintained |
| | | at 1200 ml. |

| Reactor: | ultrasound tube reactor 5GS-1.6x | | | | |
|---|---|---|---|---|---|
| | | glucose | | | |
| run time | No US | level | power | | enzyme |
| hr | Temp - F. | BGL-mg/dl | Total watts | Notes | addition |
| 0 | 71 | 0 | 235 | | |
| 0.25 | 74 | 0 | 235 | | |
| 0.68 | 87 | 5 | 217 | | |
| 0.75 | 100 | 5 | 217 | 1 | 5 |
| 1.02 | 95 | 5 | 217 | | |
| 1.42 | 88 | 5 | 212 | | |
| 1.75 | 92 | 13 | 190 | 2 | 1 |
| 2.25 | 95 | 25 | 195 | | |
| 3 | 98 | 41 | 192 | | |
| 3.03 | 99 | 42 | 210 | 4 | |
| 3.58 | 89 | 51 | 190 | 3 | 2 |
| 4.17 | 97 | 61 | 205 | | |
| 4.78 | 89 | 63 | 200 | | |
| 5.33 | 97 | 76 | 209 | | |
| 6 | 102 | 87 | 212 | | |
| 6.58 | 98 | 93 | 200 | | |
| 7.17 | 95 | 144 | 200 | 3 | 2 |
| 7.92 | 86 | 117 | 194 | | |
| 8.58 | 94 | 123 | 201 | | |
| 8.67 | 95 | 119 | 188 | | |
| 9.42 | 100 | 135 | 198 | | |
| 10.08 | 90 | 127 | 190 | 3 | 2 |
| 10.83 | 100 | 140 | 200 | | |
| 14.53 | 87 | 167 | 186 | | |
| 14.75 | 91 | 162 | 169 | 4 | |
| 15.42 | 93 | 177 | 160 | | |
| 16 | 96 | 196 | 190 | | |
| 18.17 | 94 | 212 | 156 | | |
| 18.25 | 94 | 200 | 225 | 4 | |
| 18.5 | 97 | 215 | 210 | | |
| 19.67 | 92 | 226 | 212 | | |
| 20.35 | 102 | 249 | 215 | | |
| 20.93 | 95 | 248 | 218 | | |
| 21.3 | 100 | 255 | 223 | | |
| 21.83 | 96 | 250 | 212 | 3 | 2 |
| 22.38 | 102 | 263 | 215 | | |
| 22.83 | 95 | 250 | 210 | | |
| 23.38 | 101 | 265 | 210 | | |
| 23.83 | 93 | 267 | 215 | | |
| 24.5 | 99 | 300 | 210 | | |
| 25.58 | 90 | 290 | 212 | | |
| 27.33 | 99 | 288 | 208 | | |
| 28.67 | 90 | 333 | 210 | | |
| | | | 204.0233 | | |
| | | | 94.02326 | | |

| | |
|---|---|
| applied power-110 = | 94 |
| average temp = | 95.125 |

Notes:
1. Add starting 5 ml enzyme
2. Add 1 ml enzyme
3. Add 2 ml enzyme
4. Frequency changed to balance firm volts

TABLE 4

| | | glucose | | | | |
|---|---|---|---|---|---|---|
| | Batched | level | power | Power | | |
| run time | Mixed | BGL- | Total | No | Power | |
| hr | Temp - F. | mg/dl | watts | Load W | Load W | Notes |
| 0 | 81.2 | | 402 | 127 | 402 | |
| 0.08 | 90.5 | | 408 | | 408 | 1 |
| 0.33 | 97.7 | | 403 | | 403 | |
| 0.37 | 99.8 | | 403 | | 403 | 5 |
| 0.371 | 99.8 | | 128 | 128 | | |
| 0.45 | 90.6 | | 127 | 127 | | 6 |
| 0.452 | 90.6 | | 400 | | 400 | |
| 0.9 | 99 | 12 | 412 | | 412 | 5 |
| 0.91 | 99 | | 133 | 133 | | |
| 0.98 | 89.4 | | 129 | 129 | | 6 |
| 0.981 | 89.4 | | 385 | | 385 | |
| 1.25 | 95.6 | 22 | 432 | | 432 | |
| 1.43 | 99.6 | | 417 | | 417 | 5 |
| 1.431 | 99.6 | | 130 | 130 | | |
| 1.58 | 86.8 | | 128 | 128 | | 6 |
| 1.581 | 86.8 | | 310 | | 310 | |
| 1.67 | 88.6 | | 425 | | 425 | |
| 1.8 | 94 | 32 | 433 | | 433 | |
| 1.97 | 99.7 | | 400 | | 400 | 5 |
| 1.971 | | | 135 | 135 | | |
| 2.08 | 90 | | 132 | 132 | | 6 |
| 2.081 | | | 427 | | 427 | |
| 2.2 | 95.4 | 41 | 418 | | 418 | |
| 2.33 | 100.1 | | 398 | | 398 | 5 |
| 2.331 | | | 129 | 129 | | |
| 2.45 | 90.9 | | 125 | 125 | | 6 |
| 2.451 | | | 414 | | 414 | |
| 2.73 | 101 | | 390 | | 390 | 5 |
| 2.731 | | | 130 | 130 | | |
| 2.82 | 97.3 | 54 | 133 | 133 | | 3 |
| 2.87 | 90.6 | | 129 | 129 | | 6 |
| 2.871 | | | 420 | | 420 | |
| 3.2 | 101.5 | | 408 | | 408 | 5 |
| 3.201 | | | 129 | 129 | | |
| 3.25 | 97.7 | 70 | 130 | 130 | | |
| 3.45 | 89.5 | | 130 | 130 | | 6 |
| 3.451 | | | 392 | | 392 | |
| 3.6 | 95.8 | 82 | 409 | | 409 | |
| 3.72 | 99.6 | | 397 | | 397 | 5 |
| 3.721 | | | 129 | 129 | | |
| 3.87 | 89 | | 130 | 130 | | 6 |
| 3.871 | | | 409 | | 409 | |
| 4.12 | 99 | | 386 | | 386 | 5 |
| 4.121 | | | 125 | 125 | | |
| 4.17 | 94 | 90 | 126 | 126 | | 6 |
| 4.33 | 83.2 | | 126 | 126 | | |
| 4.331 | | | 413 | | 413 | |
| 4.73 | 99.3 | 108 | 379 | | 379 | 5 |
| 4.731 | | | 124 | 124 | | |
| 4.92 | 84.1 | | 128 | 128 | | 6 |
| 4.921 | | | 412 | | 412 | |
| 5.32 | 100.1 | 113 | 399 | | 399 | 5 |
| 5.321 | | | 130 | 130 | | |
| 5.53 | 82.4 | | 127 | 127 | | 6 |
| 5.531 | | | 429 | | 429 | |
| 6.08 | 101.4 | 112 | 406 | | 406 | 5 |
| 6.081 | | | 125 | 125 | | |
| 6.23 | 86.9 | | 128 | 128 | | 6 |
| 6.231 | | | 432 | | 432 | |
| 6.42 | 95.9 | 130 | 419 | | 419 | |
| 6.6 | 100.1 | | 404 | | 404 | 5 |
| 6.601 | | | 128 | 128 | | |
| 6.7 | 90.4 | | 130 | 130 | | 6 |
| 6.701 | | | 420 | | 420 | 2 |
| 6.95 | 100.2 | | 405 | | 405 | 5 |
| 6.951 | | | 127 | 127 | | |
| 7.1 | 85.5 | 140 | 131 | 131 | | 6 |
| 7.101 | | | 458 | | 458 | |
| 7.5 | 100.2 | | 408 | | 408 | 5 |
| 7.501 | | | 128 | 128 | | |
| 7.92 | 75.2 | | 133 | 133 | | |
| 7.921 | | | 464 | | 464 | 6 |
| 8 | 83.1 | 151 | 492 | | 492 | |
| 8.2 | 95.2 | | 435 | | 435 | |
| 8.5 | 101.4 | | 411 | | 411 | 5 |
| 8.501 | 98.6 | 173 | 132 | 132 | | 2 |
| 8.67 | 85 | | 130 | 130 | | 6 |
| 8.671 | | | 419 | | 419 | |
| 9.1 | 100.7 | 183 | 408 | | 408 | 5 |
| 9.42 | 80.3 | | 127 | 127 | | |
| 9.421 | | | 424 | | 424 | 6 |
| 9.6 | 94.5 | 182 | 421 | | 421 | |
| 9.85 | 100.1 | 199 | 414 | | 414 | 5 |
| 9.851 | | | 129 | 129 | | |
| 9.95 | 90 | | 424 | | 424 | 6 |
| 10 | 92.5 | | 424 | | 424 | |
| 10.3 | 100.3 | | 399 | | 399 | 5 |
| 10.4 | 90.4 | 198 | 128 | 128 | | 6 |
| 10.401 | | | 422 | | 422 | |
| 10.5 | 96.2 | 198 | 418 | | 418 | 2 |
| 10.75 | 100.7 | | 409 | | 409 | 5 |
| 10.751 | | | 126 | 126 | | |
| 10.85 | 90.2 | | 129 | 129 | | 6 |
| 10.851 | | | 438 | | 438 | |
| 10.9 | 91.7 | | 395 | | 395 | |
| 11 | 95.2 | 154 | 392 | | 392 | |
| 11.5 | 100.4 | | 380 | | 380 | 5 |
| 11.501 | | 210 | 128 | 128 | | |
| 11.6 | 90.2 | | 123 | 123 | | 6 |
| 11.601 | | | 381 | | 381 | |
| 11.83 | 99 | 204 | 371 | | 371 | |
| 12 | | 253 | | | | |

TABLE 5

| | | glucose | | | |
|---|---|---|---|---|---|
| | Batched | level | power | | |
| run time | Mixed | BGL- | Total | | no load |
| hr | Temp - F. | mg/dl | watts | Notes | watts |
| 0 | 78 | | 256 | | 122 |
| 0.5 | 88 | | 229 | 1 | |
| 0.75 | 92.6 | | 272 | | |
| 0.84 | 96.3 | | 264 | | 118 |
| 1 | 100.6 | | 260 | | |
| 1.1 | 99.5 | | 289 | | |
| 3 | 92.9 | 63 | 288 | 3 | |
| 3.5 | 92.8 | 81 | 279 | | |
| 4 | 93 | 102 | 281 | | 117 |
| 4.5 | 92.8 | 104 | 271 | | |
| 5 | 92.8 | 116 | 268 | | |
| 5.5 | 93 | 135 | 265 | | |
| 5.75 | 92.6 | 117 | 266 | | |
| 6 | 92.6 | 127 | 265 | 2 | 115 |
| 6.5 | 92.4 | 135 | 264 | | |
| 7 | 92.2 | 150 | 269 | | |
| 8 | 92.2 | 176 | 266 | | |
| 8.5 | 92.4 | 184 | 264 | | |
| 9 | 92.8 | 186 | 260 | 2 | |
| 9.1 | 91.9 | 186 | 177 | 7 | |
| 9.5 | 92.5 | 189 | 175 | | |
| 10 | 96 | 185 | 173 | 2 | |
| 11.5 | 84.1 | 210 | 185 | | |
| 12.5 | 95.2 | 218 | 173 | | 113 |
| 13.5 | 97.9 | 250 | 169 | | |
| 15 | 99.2 | 272 | 168 | | |
| 15.5 | 87.9 | 263 | 182 | | |
| 16 | 85.9 | 245 | 186 | | |
| 16.5 | 92 | 284 | 171 | | 110 |
| 17 | 93.5 | 273 | 174 | | 107 |

What is claimed is:

1. A method for increasing enzymatic activity during a continuous processing reaction, comprising applying ultrasonic energy to the reaction.

2. The method of claim 1, wherein the enzymatic activity is derived from an enzyme selected from the group consisting of: cellulase, cellobiase and lignase.

3. The method of claim 1, wherein the enzymatic activity is that of an enzyme expressed by a microorganism.

4. The method of claim 3, wherein the microorganism from a genus selected from the group consisting of: Trichoderma, Saccharomyces, Kluyveromyces, Dekkera, Candida, Aspergillus, Microbispora, Zymomonas, Chrysosporium, Escherichia, and Clostridium.

5. The method of claim 3, wherein the microorganism is genetically modified.

6. The method of claim 5, wherein the genetically-modified microorganism expresses an exogenous enzyme.

7. The method of claim 1, wherein the ultrasonic energy is applied continuously or in pulses.

8. The method of claim 1, wherein the frequency of the applied ultrasonic energy is either fixed or cycled through a range of frequencies using a sweep oscillator.

9. A method for improving the efficiency of the saccharification of cellulosic material during continuous processing, comprising administering ultrasonic energy to a saccharification reaction.

10. A method for increasing enzyme mobility during a continuous process, comprising agitating the continuous process mixture using ultrasonic energy.
